(19) United States
(12) Patent Application Publication
(10) Pub. No.: US 2023/0068316 A1
(43) Pub. Date: Mar. 2, 2023

(54) Temperature Detection Through Differential Dual Detectors

(71) Applicant: TRINAMIX GMBH, Ludwigshafen am Rhein (DE)

(72) Inventors: Celal Mohan OEGUEN, Ludwigshafen (DE); Robert GUST, Ludwigshafen (DE)

(21) Appl. No.: 17/904,359

(22) PCT Filed: Feb. 5, 2021
(86) PCT No.: PCT/EP2021/052783

§ 371 Date: Aug. 16, 2022

(30) Foreign Application Priority Data
Feb. 6, 2020 (EP) 20155845.9

(51) Int. Cl. G01J 5/22; G01N 21/31; G01N 33/00

(52) U.S. Cl. CPC G01J 5/22; G01N 21/31; G01N 33/0027

(57) ABSTRACT

Disclosed herein is a sensor system including four interconnected resistors, where two of the resistors are photoconductive detectors, where the photoconductive detectors are illuminated with light at least at two different wavelengths, where two of the resistors does not change their resistance due to the illumination, where an external voltage is applicable to the sensor system, where a differential voltage is measurable, which depends on the resistance changes of the illuminated photoconductive detectors, where the differential voltage gives a mathematical ratio of the four respective resistances.

### FIELD OF THE INVENTION

The invention relates to an electronic read-out device for differential measurement of the changing resistance of a pair of photoconductors, also known as photo resistive detectors.

### PRIOR ART

Photoconductors are sensors, which require an external excitation signal to generate an electrical output depending on the measured physical quantity. In the case of photoconductors is this physical quantity the illumination. Most commonly, a voltage VBias is applied to the photoconductors as excitation signal.

Used with specific filters photoconductors can be used as infrared thermometers by inferring temperature from a portion of the thermal radiation emitted by the object being measured.

Dual-wave IR measurement, which allows the determination of the temperature without knowing the emissivity of the measurement object, is a known approach and mentioned in the literature for applications in a variety of industries.

The paper Sensing Systems for Glass Ceramic Cooktops; by Joseph Paradiso, Lance Borque, Philip Bramson, Mat Laibowitz, Hong Ma, Mat Malinowski edited by Responsive Environments Group MIT Media Lab in Jul. 18, 2003, gives an overview of measurement systems used in Schotts Ceramic Glass Cooktops. A number of sensing material, electric circuits, and measurement strategies have already been executed. (https://resenv.media.mit.edu/pubs/papers/Sensing %20Systems %20forCooktop1.pdf)

The United States Consumer Product Safety Commission had to discuss the measurement of temperature on glass ceramic cooktops in 2002. Their memorandum can be found https://www.cpsc.gov/s3fs-public/pdfs/ceramic.PDF

Enhanced multi-wavelength sensors based on MEMS technology are also commercially available.

The output of optical detectors is recorded with an adequate signal processing circuit and afterwards by means of analog digital converters. The digital values can then be used to calculate quotients, which depends on the temperature of the measured object. An adequate Look-up table can be used to convert the quotient into temperature.

For this approach, two read-out electronics are required, and the calculation of the quotient is performed by means of a microcontroller or a similar digital signal processing unit. For high accuracy measurement, the required components for the read-out electronics are expensive and every channel (in the simplest approach two channels for dual wavelength) costs more money.

Furthermore, the temperature dependency of the detectors should be compensated by measuring the temperature of the detectors and once again by performing digital calculations on the microcontroller. Alternatively, a darkened detector can be positioned on the same substrate as the other detectors in thermal equilibrium with each other, and by monitoring the thermal drift of the darkened detector. Since the darkened detector does not see the radiation from the measured object, the only signal change it produces is due to the thermal drift. Yet, another costly read-out electronics is required for the darkened detector.

The remote temperature measurement requires previous knowledge about the emissivity of the measured object. Different types of objects cannot be measured correctly without a repetitive adjustment of the measurement settings because of difference of their respective emissivity.

Emissivity independent temperature measurement can be realized by employing multiple sensors at different wavelengths and then combining their values. This approach increases the material costs of the measurement setup. With this approach thermal drift of the detectors should also be taken into account by adding further detectors and read-out electronics.

The calculation of the quotient and the compensation of the temperature drift on an analog basis with single read-out electronics is required.

### Problem Addressed by the Invention

Therefore, a problem addressed by the present invention is that of specifying a device and methods which at least substantially avoid the disadvantages of known circuits of this type. In particular, a simplified solution to measure the temperature of an object without any knowledge about its emissivity with only one read-out electronics would be desirable.

### SUMMARY OF THE INVENTION

This problem is solved by the invention with the features of the independent patent claims. Advantageous developments of the invention, which can be realized individually or in combination, are presented in the dependent claims and/or in the following specification and detailed embodiments.

As used herein, the expressions “have”, “comprise” and “contain” as well as grammatical variations thereof are used in a non-exclusive way. Thus, the expression “A has B” as well as the expression “A comprises B” or “A contains B” may both refer to the fact that, besides B, A contains one or more further components and/or constituents, and to the case in which, besides B, no other components, constituents or elements are present in A.

In a first aspect of the invention, a sensor system is disclosed. The sensor system comprises four interconnected resistors. At least two of the resistors are photoconductive detectors configured for each exhibiting an electrical resistance dependent on an illumination of its respective light sensitive region. The photoconductive detectors are illuminated with light at least at two different wavelengths wherein at least two of the photoconductive detectors each respond to electromagnetic energy of a different wavelength. The two other resistors are configured for each exhibiting an electrical resistance essentially constant under illumination. An external voltage is applicable to the sensor system such as by using at least one voltage source. The sensor system is configured for measuring a differential voltage. The differential voltage is dependent on changes of the electrical resistances of the photoconductive detectors. The differential voltage gives a mathematical ratio of the four respective resistances.

The sensor system according to the invention is based on at least two photoconductive detectors, whereas they are illuminated at least at two different wavelengths. Two further temperature sensitive resistors, such as thermistors, are required for temperature drift compensation, whereas they exhibit the same temperature-resistance behavior as the photoconductive detectors. For practical reasons, instead of thermistors, two additional photoconductive detectors may be employed, whereas they are darkened, which means that they are not illuminated. A differential voltage depending on the resistance changes of the illuminated detectors is generated, whereas the differential voltage gives a mathematical ratio of the four detector signals.

The term “system” as used herein is a broad term and is to be given its ordinary and customary meaning to a person of ordinary skill in the art and is not to be limited to a special or customized meaning. The term specifically may refer, without limitation, to an arbitrary set of interacting or interdependent components parts forming a whole. Specifically, the components may interact with each other in order to fulfill at least one common function. The at least two components may be handled independently or may be coupled or connectable. The term “sensor system” as used herein is a broad term and is to be given its ordinary and customary meaning to a person of ordinary skill in the art and is not to be limited to a special or customized meaning. The term specifically may refer, without limitation, to a system comprising at least sensors, in particular at least two photoconductive detectors.

The term “photoconductive detector”, also denoted photoconductor, as used herein is a broad term and is to be given its ordinary and customary meaning to a person of ordinary skill in the art and is not to be limited to a special or customized meaning. The term specifically may refer, without limitation, to a light sensitive element capable of exhibiting a specific electrical resistance Rₚₕₒₜₒ dependent on an illumination of the light-sensitive region the photoconductor. Specifically, the electrical resistance is dependent on the illumination of a material of the photoconductive detector. The photoconductive detectors each may comprise a light-sensitive region comprising a “photoconductive material”. A photoconductive detector can, for example, be applied in light-sensitive detector circuits. Each of the photoconductive detectors may be configured for exhibiting an electrical resistance dependent on an illumination of its light-sensitive region.

The photoconductive detectors may be arranged in at least one array of photoconductors, in particular next to each other. The photoconductive detectors may be neighboring detectors of the array. However, embodiments are possible in which additional photoconductive detectors are present between the photoconductive detectors. The term “array” of photoconductive detectors as used herein is a broad term and is to be given its ordinary and customary meaning to a person of ordinary skill in the art and is not to be limited to a special or customized meaning. The term specifically may refer, without limitation, to a plurality of photoconductors arranged in a matrix having a plurality of pixels. As further used herein, the term “matrix” generally refers to an arrangement of a plurality of elements in a predetermined geometrical order. The matrix specifically may be or may comprise a rectangular matrix having one or more rows and one or more columns. The rows and columns specifically may be arranged in a rectangular fashion. It shall be outlined, however, that other arrangements are feasible, such as nonrectangular arrangements. As an example, circular arrangements are also feasible, wherein the elements are arranged in concentric circles or ellipses about a center point. For example, the matrix may be a single row of pixels. Other arrangements are feasible. The photoconductive detectors of the matrix specifically may be equal in one or more of size, sensitivity and other optical, electrical and mechanical properties. The light-sensitive regions of all photoconductive detectors of the matrix specifically may be located in a common plane, such that a light beam illuminating the array may generate a light spot on the common plane. The array may be fabricated monolithically on the same substrate. The photoconductive detectors of the array may be designed identical, in particular with respect to size and/or shape of their light-sensitive regions and/or photoconductive materials.

The term “illumination” as used herein is a broad term and is to be given its ordinary and customary meaning to a person of ordinary skill in the art and is not to be limited to a special or customized meaning. The term specifically may refer, without limitation, to electromagnetic radiation in one or more of the visible spectral range, the ultraviolet spectral range and the infrared spectral range. Therein, in partial accordance with standard ISO-21348, the term visible spectral range generally refers to a spectral range of 380 nm to 760 nm. The term infrared (IR) spectral range generally refers to electromagnetic radiation in the range of 760 nm to 1000 μm, wherein the range of 760 nm to 1.4 μm is usually denominated as the near infrared (NIR) spectral range, and the range from 15 μm to 1000 μm as the far infrared (FIR) spectral range. The term “ultraviolet spectral range” generally refers to electromagnetic radiation in the range of 1 nm to 380 nm, preferably in the range of 100 nm to 380 nm. In the following, the term “illumination” is also denoted as “light”. Preferably, illumination as used within the present invention is visible light, i.e. light in the visible spectral range, and/or infrared light, i.e. light in the infrared spectral range.

As used herein, the term “light-sensitive region” generally refers to an area of the photoconductor being sensitive to an illumination, e.g. by an incident light beam. For example, the light-sensitive region may be a two-dimensional or three-dimensional region which preferably, but not necessarily, is continuous and can form a continuous region. The photoconductive detectors can have one or else a plurality of such light-sensitive regions. As used herein, the term “to exhibit an electrical resistance dependent on an illumination” generally refers to that the electrical resistance of the photoconductive detectors is adjusted and/or changed and/or varied dependent, on the illumination, in particular an intensity of the illumination, of the light-sensitive region. In particular, in response to the illumination, the electrical resistance is adjusted and/or changed and/or varied. When the photoconductive detector is illuminated the photoconductive detector may exhibit a decrease in electrical resistance. The photoconductive detector may lower its resistivity when illuminated. Specifically, the electrical resistance of the photoconductor may decrease with increasing incident light intensity. The change between dark resistance and bright resistance is the quantity to be measured or to be read out, and may be denoted as output current of the photoconductive detectors. As used herein, the term “dark resistance” generally refers to an electrical resistance of the photoconductive detector in unlit state, i.e. without illumination. As further used herein, the term “bright resistance” refers to an electrical resistance of the photoconductive detector under illumination.

The photoconductive detector may comprise at least one photoconductive material. Since an electrical resistance is defined as the reciprocal value of the electrical conductivity, alternatively, the term “photoresistive material” may also be used to denominate the same kind of material. The light-sensitive region may comprise at least one photoconductive material selected from the group consisting of lead sulfide (PbS); lead selenide (PbSe); mercury cadmium telluride (HgCdTe); cadmium sulfide (CdS); cadmium selenide (CdSe); indium antimonide (InSb); indium arsenide (InAs); indium gallium arsenide (InGaAs); extrinsic semiconductors, e.g. doped Ge, Si, GaAs, organic semiconductors. However, other materials may be feasible. Further possible photoconductive materials are described in WO 2016/120392 A1, for example. For example, the photoconductive detector may be a photoconductor commercially available under the brand name Hertzstueck™ from trinamiX GmbH, D-67056 Ludwigshafen am Rhein, Germany.

For example, the light-sensitive region may be illuminated by at least one illumination source. The sensor system may comprise the at least one illumination source. The illumination source can for example be or comprise an ambient light source and/or may be or may comprise an artificial illumination source. By way of example, the illumination source may comprise at least one infrared emitter and/or at least one emitter for visible light and/or at least one emitter for ultraviolet light. By way of example, the illumination source may comprise at least one light emitting diode and/or at least one laser diode. The illumination source can comprise in particular one or a plurality of the following illumination sources: a laser, in particular a laser diode, although in principle, alternatively or additionally, other types of lasers can also be used; a light emitting diode; an incandescent lamp; a neon light; a flame source; an organic light source, in particular an organic light emitting diode; a structured light source. Alternatively or additionally, other illumination sources can also be used. The illumination source generally may be adapted to emit light in at least one of: the ultraviolet spectral range, the infrared spectral range. Most preferably, at least one illumination source is adapted to emit light in the NIR and IR range, preferably in the range of 800 nm and 5000 nm, most preferably in the range of 1000 nm and 4000 nm.

The illumination source may comprise at least one non-continuous light source. Alternatively, the illumination source may comprise at least one continuous light source. The light source may be an arbitrary light source having at least one radiating wavelength having an overlap to the sensitive wavelength of the photoconductor. For example, the light source may be configured for generating a Planckian radiation. For example, the light source may comprise at least one light emitting diode (LED) and/or at least one Laser source. For example, the light source may be configured for generating illumination by an exotherm reaction, like an oxidation of liquid or solid-material or Gas. For example, the light source may be configured for generating illumination out of fluorescent effects. The illumination source may be configured for generating at least one modulated light beam. Alternatively, the light beam generated by the illumination source may be non-modulated and/or may be modulated by further optical means. The illumination source may comprise at least one optical chopper device configured for modulating a light beam from the continuous light source. The optical chopper device may be configured for periodically interrupting the light beam from the continuous light source. For example, the optical chopper device may be or may comprise at least one variable frequency rotating disc chopper and/or at least one fixed frequency tuning fork chopper and/or at least one optical shutter. Due to the non-continuous illumination the output current may be a changing current signal, also denoted modulation current. The modulated current may be small comparted to dark current of the photoconductive detector.

The photoconductive detectors each respond to electromagnetic energy of a different wavelength. The present invention proposes dual-wavelength, in particular infrared measurement, by means of the photoconductive detectors configured for being sensitive at at least two different wavelengths. In particular, the photoconductive detectors each may detect electromagnetic absorption at different wavelengths in the electromagnetic spectrum. The photoconductive detectors of the array may be designed such that each pixel in the array responds to electromagnetic energy of a different wavelength. The photoconductive detectors may be covered by filter elements, also denoted as filters, for preparation of illumination at different wavelengths. For example, at least one filter arrangement may be used. However, other arrangements are possible. This may allow using the array for spectrometer applications.

The sensor system, in particular the photoconductive detectors, more particular their light sensitive regions, may be arranged in direct line of sight of an object to be measured. The filter elements may be arranged to be within the wavelength range of the electromagnetic radiation which is in the line of sight. The sensor system and the measured object may be separated by a separating object, such as a separating objected comprised by the sensor system. The separating object may be at least partially transparent at the at least two wavelengths to which the two photoconductive detectors are responsible. The filters may be arranged to be within the wavelength range of the electromagnetic radiation transmitted through the separating object.

The sensor system may comprise at least one bias voltage source configured for applying at least one bias voltage to the photoconductive detectors. The photoconductive detectors may be electrically connected with the bias voltage source. As used herein, the term “bias voltage source” refers to at last one voltage source configured for generating the bias voltage. The bias voltage may be the voltage applied across the photoconductor material. The photoconductive detectors each may be connected to the bias voltage source such that the bias voltage source can apply the bias voltage to the photoconductive detectors.

The term “essentially constant under illumination” as used herein is a broad term and is to be given its ordinary and customary meaning to a person of ordinary skill in the art and is not to be limited to a special or customized meaning. The term specifically may refer, without limitation, to embodiments in which the resistance is constant, wherein deviations are possible below 5%, preferably below 1%, more preferably below 0.1%. The resistors exhibiting an electrical resistance essentially constant under illumination are not responding to the illumination. For example, the resistors exhibiting an electrical resistance essentially constant under illumination may be photoconductive detectors darkened by a cover. Thus, the resistors may be covered so they don't see any irradiation. A change on their output signal may depend on their temperature drift.

The resistors essentially constant under illumination may be temperature sensitive resistors. For example, the resistors essentially constant under illumination may be thermistors. A change of their resistance as a function of temperature may have the same characteristics as of the photoconductive detectors. The temperature sensitive resistors may exhibit the same temperature-resistance behavior as the photoconductive detectors. Thus, the temperature sensitive resistors can be used for temperature drift compensation.

The sensor system is configured for measuring a differential voltage. The differential voltage is dependent on changes of the electrical resistances of the photoconductive detectors. The term “measuring a differential voltage” as used herein is a broad term and is to be given its ordinary and customary meaning to a person of ordinary skill in the art and is not to be limited to a special or customized meaning. The term specifically may refer, without limitation, to determine differences, in particular changes, between voltages, in particular across the photoconductive detectors such as at different time points and/or illumination states. The differential voltage gives a mathematical ratio of the four respective resistances.

The term “interconnected resistors” as used herein is a broad term and is to be given its ordinary and customary meaning to a person of ordinary skill in the art and is not to be limited to a special or customized meaning. The term specifically may refer, without limitation, to arrangement that each of the resistors is connected to at least two other resistors of the system. For example, the resistors may be interconnected by a bridge circuit arrangement. For example, the bridge circuit arrangement may comprise at least one Wheatstone bridge. The term “Wheatstone bridge” as used herein is a broad term and is to be given its ordinary and customary meaning to a person of ordinary skill in the art and is not to be limited to a special or customized meaning. The term specifically may refer, without limitation, to an electrical circuit configured for determining an unknown electrical resistance by balancing two legs of a bridge circuit, wherein, usually, one of the legs comprises the unknown electrical resistance. For example, the Wheatstone bridge may comprise the four interconnected resistors, the photoconductive detectors Rₚₕₒₜₒ₁ and Rₚₕₒₜₒ₂ configured for each exhibiting an electrical resistance dependent on an illumination of its respective light sensitive region, and two other resistors R₃ and R₄.

The sensor system may comprise a supply voltage source configured for applying a supply voltage Vₛ, such as a direct current (DC) voltage or an alternating current (AC) voltage, to the Wheatstone Bridge. Therefore, the Wheatstone Bridge may be connected to the supply voltage source.

The differential voltage V_{Diff} can be calculated directly by means of the Wheatstone bridge as given in the following equation:

$${V}_{Diff} = {V}_{S} \frac{{R}_{P hoto 1} \cdot {R}_{3} - {R}_{P hoto 2} \cdot {R}_{1}}{( {R}_{P hoto 1} + R 1 ) \cdot ( {R}_{P hoto 2} + R 3 )}$$

Sourced by the supply voltage Vₛ, the circuit has two symmetric legs of a bridge consisting of two non-photosensitive resistors R₁ and R₃ and one photosensitive resistor R_{Photo1} or R_{Photo2}. Thus, the measurement of V_{Diff} can be used to calculate the quotient of the values, measured at different wavelengths on an analog basis.

The resistors R₁ and R₃ may be photoconductive detectors darkened by a cover. In particular, the photoconductive detectors darkened by a cover may be similar to the non-covered photoconductive detectors such as with the identical physical properties such as electrical, optical, opto-electrical and mechanical properties. In particular, the photoconductive detectors darkened by a cover may be similar to the non-covered photoconductive detectors such as from the identical manufacturer, available under the identical product number and the like. A change on their output signal may depend on their temperature drift. The temperature drift of the non-covered photoconductive detectors may be similar or identical. With the proposed circuit, any temperature drift of the photoconductive detectors automatically may be corrected by the Wheatstone bridge, as long as the photoconductive detectors, darkened or illuminated, exhibit same temperature behavior.

The sensor system furthermore may comprise at least one evaluation device configured for determining an output signal of at least one output of the photoconductive detectors. The evaluation device may be configured for determining an illumination intensity by evaluating the output signal. As used herein, the term “evaluation device” generally refers to an arbitrary device designed to determine and/or generating at least one voltage output signal at the voltage output. As an example, the evaluation device may be or may comprise one or more integrated circuits, such as one or more application-specific integrated circuits (ASICs), and/or one or more data processing devices, such as one or more computers, preferably one or more microcomputers and/or microcontrollers. Additional components may be comprised, such as one or more preprocessing devices and/or data acquisition devices, such as one or more devices for receiving and/or preprocessing of the voltage signal, such as one or more AD-converters and/or one or more filters. Further, the evaluation device may comprise one or more data storage devices. The evaluation device may comprise one or more interfaces, such as one or more wireless interfaces and/or one or more wire-bound interfaces. The evaluation device may particularly comprise at least one data processing device, in particular an electronic data processing device, which can be designed to determine at least one output voltage signal. The evaluation device can also be designed to completely or partly control the at least one illumination source and/or to control the at least one voltage source and/or to adjust the at least one load resistor. The evaluation device may further comprise one or more additional components, such as one or more electronic hardware components and/or one or more software components, such as one or more measurement units and/or one or more evaluation units and/or one or more controlling units. For example, the evaluation device may comprise at least one measurement device adapted to measure the at least one output voltage signal, e.g. at least one voltmeter. The evaluation device may be configured for performing one or more operations of the group consisting of: at least one Fourier transformation; a counting of frequency, an edge detection, a measurement of the period length and the like.

Depending on the setup, direct measurement of the infrared radiation can be difficult. For example, cooktops made of ceramic glass are to separate the electrical, or fuel driven heat source from the pots and pans for hygienic reason. The cooktop glass is at least partially transparent for electromagnetic radiation at wavelengths between 1 and 2.7 μm. At lower temperature (˜80° C.-100° C., e.g. about boiling temperature of water), the infrared radiation is very weak in the near infrared range (IR-A up to 1.4 μm). It makes the use of photovoltaic detectors like extended-InGaS very limited due to their spectral sensitivity range. The high cost of extended-InGaS detectors makes their use as multi- or dual wavelength sensors unfeasible.

Since the radiation which is emitted by the measured object is not modulated, temperature sensors based on the pyroelectric effect cannot be used for this measurement due to their physical properties. For this purpose, mechanical or optical choppers are required, which increase the complexity and the price of the measurement setup, while decreasing the life span.

Thermopiles offer a cheap alternative with their broad band spectral sensitivity and ability to detect unmodulated radiation, yet their detectivity are very low compared to quantum detectors, such as photovoltaic and photoconductive detectors. Thus, the achievable resolution is relatively low.

Photoconductive detectors offer good detectivity and can be employed also for unmodulated thermal radiators. Yet, photoconductors require an external excitation signal to generate an electrical output depending on the measured physical quantity. In the case of photoconductors this physical quantity is the luminous strength. Most commonly, a voltage V_{Bias} is applied to the photoconductors as excitation signal.

The photoconductors change their resistance depending on the illumination. The change itself is relatively small compared to the total resistance value of the photoconductor. As an example, a PbS-detector with dimension of 2 mm×2 mm featuring a resistance of about 1 MΩ changes its resistance due to infrared radiation at 1550 nm with an irradiance of 16 μW/cm2 about 10 kΩ, which corresponds 1% change. Thus, the excitation signal will be orders of magnitude greater than the electrical output change due to the illumination. Without any filtering, the read-out electronics should be able to measure the whole signal but still solve the change of 1% with a relatively good resolution. Such read-out electronics are commercially available, yet very expensive.

The photoconductors are commonly measured by means of a voltage divider, which applies a constant DC bias voltage to the photoconductor. Any instability or deviation of the DC voltage directly affects the output signal and lead to measurement errors. Additionally, the 1/f noise depends on I_{DC}, the DC part of the current flowing through the detector. Thus, a constant DC voltage as bias is also disadvantageous.

Also, any change or fluctuation in the supply voltage leads to a measurement error. Thus, only very low noise supply sources, such as batteries, can be used for high precision measurements.

Summarizing, in the context of the present invention, the following embodiments are regarded as particularly preferred:
Embodiment 1. A sensor system, comprising of four interconnected resistors, whereas, two of the resistors are photoconductive detectors, whereas the photoconductive detectors are illuminated with light at least at two different wavelengths, whereas two of the resistors does not change their resistance due to the illumination, whereas an external voltage is applicable to the sensor system, whereas a differential voltage is measurable, whereas the differential voltage is dependent on the resistance changes of the illuminated photoconductive detectors, whereas the differential voltage gives a mathematical ratio of the four respective resistances.
Embodiment 2. A sensor system according to the preceding embodiment comprising, the resistors, which are not responding to the illumination, are photoconductive detectors, darkened by a cover.
Embodiment 3. A sensor system according to the preceding embodiments comprising, the photoconductive resistors are covered by filter elements for preparation of light at different wavelengths.
Embodiment 4. A sensor system according to any one of the preceding embodiments comprising, the photoconductive resistors are interconnected by a bridge circuit arrangement.
Embodiment 5. A sensor system according to any one of the preceding embodiments whereas the sensor system is in direct line of sight of the measured object, whereas filters are arranged to be within the wavelength range of the electromagnetic radiation which is in the line of sight.
Embodiment 6. A sensor system according to any one of the preceding embodiments whereas the sensor system and measured object is separated by another object, whereas the separating object is at least partially transparent at least at two wavelengths, whereas the filters are arranged to be within the wavelength range of the electromagnetic radiation transmitted through the separating object.
Embodiment 7. A sensor system according to any one of the preceding embodiments whereas the four photoconductive resistors are arranged in an array next to another.
Embodiment 8. Use of a sensor system according to any one of the preceding embodiments as a sensor for temperature measurement.
Embodiment 9. Use of a sensor system according to any one of the preceding embodiments as a sensor for gas and/or liquid analysis.
Embodiment 10. Use of a sensor system according to any one of the preceding embodiments as a sensor for concentration of gas and/or liquid or gases and/or liquids.
Embodiment 11. Use of a sensor system according to any one of the preceding embodiments as a sensor for material classification between to pre-defined material classes.

### BRIEF DESCRIPTION OF THE FIGURES

Further optional details and features of the invention are evident from the description of preferred exemplary embodiments which follows in conjunction with the dependent claims. In this context, the particular features may be implemented alone or with features in combination. The invention is not restricted to the exemplary embodiments. The exemplary embodiments are shown schematically in the figures. Identical reference numerals in the individual figures refer to identical elements or elements with identical function, or elements which correspond to one another with regard to their functions.

Specifically, in the figures:

FIG. 1 illustrates a first electrical circuit to measure electrical resistance by a Wheatstone Bridge;

FIG. 2 illustrates a second electrical circuit to measure electrical resistance by a Wheatstone Bridge;

FIG. 3 illustrates a third electrical circuit to measure electrical resistance by a Wheatstone Bridge;

FIG. 4 results of a simulation for two different circuits

### EXEMPLARY EMBODIMENTS

This invention offers a simplified solution to measure the temperature of an object without any knowledge about its emissivity with only one read-out electronics. Dual-wavelength IR measurement is performed by means of the photoconductive detectors at different wavelengths.

Depending on the temperature of the measurement object, suitable photoconductors and wavelengths should be chosen.

As an example, the temperature range between 100° C. and 250° C. can be measured with 2 mm×2 mm PbS detectors, while sampling the wavelength ranges between 2.2 and 2.4 um with one detector and the range between 2.6 and 2.8 um with the other. Suitable optical filters can be positioned on top of the optical detectors, thus sampling the chosen wavelength ranges.

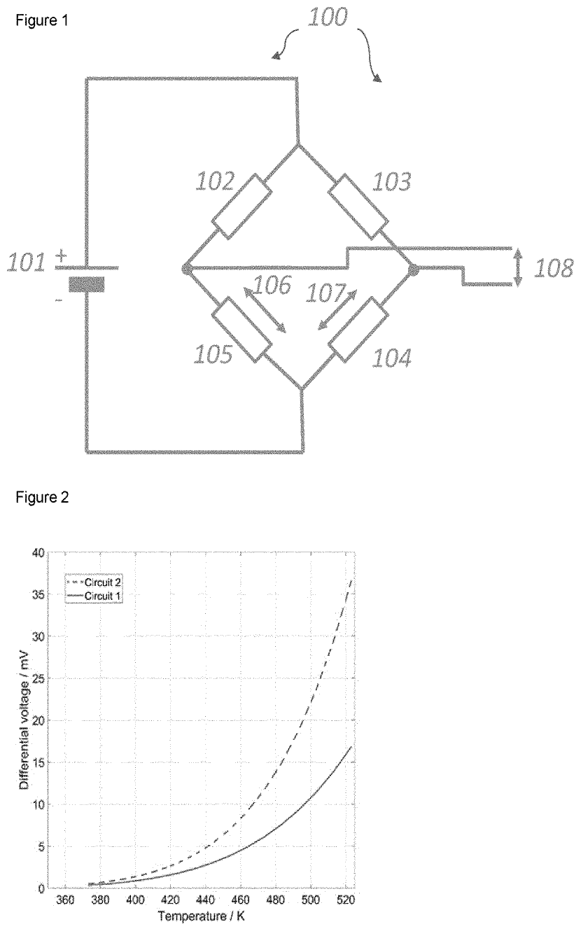

FIG. 1 shows an embodiment of a sensor system 100 according to the present invention. The Sensor system 100 comprises four interconnected resistors 102, 103, 104, 105. At least two of the resistors, in FIG. 1 resistors 104 and 105, are photoconductive detectors R_{Photo1} and R_{Photo2} configured for each exhibiting an electrical resistance dependent on an illumination of its respective light sensitive region. At least two of the photoconductive detectors each respond to electromagnetic energy of a different wavelength. The photoconductive detectors may be arranged in at least one array of photoconductors, in particular next to each other. The photoconductive detectors may be neighboring detectors of the array. The photoconductive detectors each respond to electromagnetic energy of a different wavelength. The present invention proposes dual-wavelength, in particular infrared measurement, by means of the photoconductive detectors configured for being sensitive at at least two different wavelengths. In particular, the photoconductive detectors each may detect electromagnetic absorption at different wavelengths in the electromagnetic spectrum. The photoconductive detectors of the array may be designed such that each pixel in the array responds to electromagnetic energy of a different wavelength. The photoconductive detectors may be covered by filter elements, also denoted as filters, for preparation of illumination at different wavelengths. For example, at least one filter arrangement may be used. However, other arrangements are possible. This may allow using the array for spectrometer applications.

The sensor system 100, in particular the photoconductive detectors, more particular their light sensitive regions, may be arranged in direct line of sight of an object to be measured. The filter elements may be arranged to be within the wavelength range of the electromagnetic radiation which is in the line of sight. The sensor system 100 and the measured object may be separated by a separating object, such as a separating objected comprised by the sensor system. The separating object may be at least partially transparent at the at least two wavelengths to which the two photoconductive detectors are responsible. The filters may be arranged to be within the wavelength range of the electromagnetic radiation transmitted through the separating object.

The sensor system 100 may comprise at least one bias voltage source configured for applying at least one bias voltage 106 and 107 to the photoconductive detectors. The photoconductive detectors may be electrically connected with the bias voltage source. The bias voltage may be the voltage applied across the photoconductor material. The photoconductive detectors each may be connected to the bias voltage source such that the bias voltage source can apply the bias voltage 106 and 107 to the photoconductive detectors.

The two other resistors R₁ and R₃, in FIG. 1 resistors 102 and 103, are configured for each exhibiting an electrical resistance essentially constant under illumination. The resistors exhibiting an electrical resistance essentially constant under illumination are not responding to the illumination. For example, the resistors exhibiting an electrical resistance essentially constant under illumination may be photoconductive detectors darkened by a cover. Thus, the resistors may be covered so they don't see any irradiation. A change on their output signal may depend on their temperature drift.

An external voltage, in particular a supply voltage, is applicable to the sensor system 100. The sensor system 100 may comprise the supply voltage source configured for applying the supply voltage Vₛ, such as a direct current (DC) voltage or an alternating current (AC) voltage, to resistors. Therefore, the resistors may be connected to the supply voltage source.

The sensor system 100 is configured for measuring a differential voltage. The differential voltage is dependent on changes of the electrical resistances of the photoconductive detectors. The differential voltage gives a mathematical ratio of the four respective resistances.

For example, the resistors may be interconnected by a bridge circuit arrangement. For example, the bridge circuit arrangement may comprise at least one Wheatstone bridge. The Wheatstone bridge may be or may comprise an electrical circuit configured for determining an unknown electrical resistance by balancing two legs of a bridge circuit, wherein one of the legs comprises the unknown electrical resistance. For example, the Wheatstone bridge may comprise the four interconnected resistors, the photoconductive detectors Rₚₕₒₜₒ₁ and Rₚₕₒₜₒ₂ configured for each exhibiting an electrical resistance dependent on an illumination of its respective light sensitive region, and two other resistors R₃ and R₄.

The quotient, in particular the differential voltage V_{Diff}, is calculated directly by means of the Wheatstone bridge as given in the following equation for the Circuit as shown in FIG. 1, resulting in a differential voltage V_{Diff} (108):

$${V}_{Diff} = {V}_{S} \frac{{R}_{P hoto 1} \cdot {R}_{3} - {R}_{P hoto 2} \cdot {R}_{1}}{( {R}_{P hoto 1} + R 1 ) \cdot ( {R}_{P hoto 2} + R 3 )}$$

Sourced by the supply voltage Vₛ (101), the circuit has two symmetric legs of a bridge consisting of two non-photosensitive resistors R₁ or R₃ (102, 103) and one photosensitive resistor R_{Photo1} or R_{Photo2} (104, 105). Thus, the measurement of V_{Diff} (108) is used to calculate the quotient of the values, measured at different wavelengths on an analog basis.

The resistors R₁ and R₃ may be darkened photoconductors, which means they are covered so they don't see any irradiation. The change on their output signal depends on their temperature drift. With the proposed circuit, any temperature drift of the detectors automatically corrected by the Wheatstone bridge, as long as the detectors, darkened or illuminated, exhibit same temperature behavior.

The Wheatstone bridge can be driven also with AC voltage, which means the bias voltage applied on the photoconductors is modulated. The modulation can be unipolar or bipolar. The frequency of the modulation can be chosen freely, but higher frequencies are recommended for low 1/f noise.

FIG. 2 shows a calculation for an example for an isotropic radiator with a 1 mm×1 mm area and with an emissivity of 1, the detectors R_{Photo1} and R_{Photo2} with bandpass filters in the above-mentioned wavelength ranges will change their resistance values differently. With 1 MΩ dark resistance for all detectors, two illuminated and two darkened, and in a distance of 10 cm from the isotropic radiator, the differential voltage can be measured as a function of temperature. Vₛ for this calculation is set 1 V. The calculated values may vary depending on the distance, emissivity of the radiator, spectral detectivity and responsivity of the detectors, the transmission properties of the used filters and many other parameters. The circuit of FIG. 1 is not the only possible solution but should serve as an example.

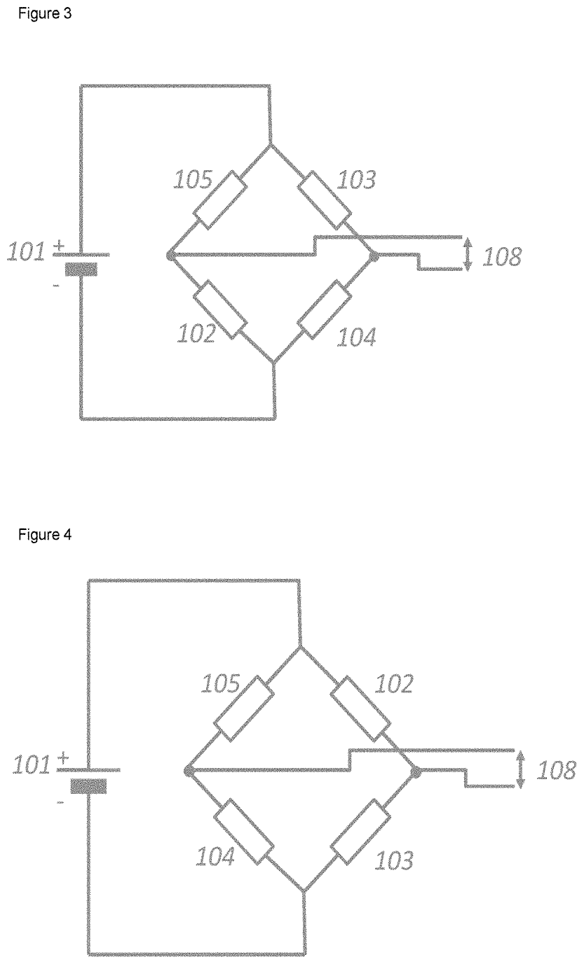

The lower curve represents the electrical circuit represented in FIG. 1. A second simulation referring the circuit of FIG. 3 is represented in the upper curve.

As long as the resistance of the photoconductors changes with the same factor due to the temperature, the differential voltage curve remains the same. Alternatively, temperature sensitive resistors can be employed as R₁ and R₃, as long as their temperature-resistance behavior is identical to that of photoconductors. It is a known fact that not only the resistance but also the responsivity of the photoconductors depends on the temperature. In this case, (if the change in the differential voltage is unacceptable high) a contact temperature sensor, such as a cheap PT100 or PT1000, can be used to correct the look-up table to convert the differential voltage into temperature.

FIG. 3 gives an alternative setting for the circuit, where the sensitivity of the circuit on the irradiance can be improved by positioning the both darkened and illuminated photoconductive detectors diagonally. Alternatively, temperature sensitive resistors can be employed as R₁ and R₃. The comparison of the resulting differential voltages can be seen in FIG. 2 upper line.

$${V}_{Diff} = {V}_{S} \frac{{R}_{P hoto 1} \cdot {R}_{3} - {R}_{P hoto 2} \cdot {R}_{1}}{( {R}_{P hoto 1} + R 1 ) \cdot ( {R}_{P hoto 2} + R 3 )}$$

The third alternative is shown in FIG. 4. The robustness of the circuit on the resistance changes may be improved by positioning the both darkened and illuminated photoconductive detectors on the same leg, respectively. The illuminated detectors can change their resistance with the same factor, while the darkened detectors, or alternatively temperature sensitive resistors, have the same change factor. The resulting differential voltage remains the same.

$${V}_{Diff} = {V}_{S} \frac{{R}_{P hoto 1} \cdot {R}_{3} - {R}_{P hoto 2} \cdot {R}_{1}}{( {R}_{P hoto 1} + R 1 ) \cdot ( {R}_{P hoto 2} + R 3 )}$$

Fluctuations on the supply voltage are balanced out since both arms of Wheatstone bridge are connected to the same potential and fluctuate the same, thus differential voltage remains constant.

By measuring the differential voltage V_{Diff} only, a detector-temperature independent, emissivity independent dual-wavelength temperature measurement can be achieved with high resolution and with minimum numbers of components for the read-out electronics. The differential voltage can then be amplified and converted into digital values by means of an ADC. There are off-the-shelf amplifiers, analog front ends and analog-digital converters available for the measurement of differential voltages for both DC and AC supply voltages Vₛ.

The sensor system 100 can be employed for emissivity independent temperature measurement. The sensor system 100 may be in the direct line of sight of the measured object or the sensor can measure the temperature of the object through another object, which is transparent at the sampled wavelengths. This is possible for the example of ceramic cooktops which are transparent for some specific infrared frequencies.

Alternatively, the sensor system 100 can be employed for gas analyses. The concentration of a gas can be determined by measuring the decrease of the light intensity from a light source through a gas filled optical path according to Lambert-Beer law, whereas the wavelengths to be sampled should be chosen depending on the gas to be measured. Generally, two wavelengths are chosen in such a way, that the measured gas absorbs at one wavelength and transmits at the other wavelength without absorption losses, thus the latter serves as the reference. The quotient of both signals depends on the measured gas concentration. In an analogous manner, liquids can also be monitored.

The sensor system 100 can be employed for measuring the diffuse reflection from a solid, illuminated with a light source. By sampling the diffuse reflection at two wavelengths concentration of known materials can be determined, or material classification between two pre-defined classes can be performed, like human skin or not, plastic or glass etc. Such measurements are common for optical sorting tasks for the recycling of plastics, glasses etc.

### LIST OF REFERENCE NUMBERS

100 Sensor system
101 Voltage supply Vₛ
102 Resistor R₁
103 Resistor R₃
104 Photo Resistor R_{Photo1}
105 Photo Resistor R_{Photo2}
106 V_{Bias1}
107 V_{Bias2}
108 V_{Diff}

1. A sensor system comprising four interconnected resistors, wherein at least two of the resistors are photoconductive detectors configured for each exhibiting an electrical resistance dependent on an illumination of its respective light sensitive region, wherein at least two of the photoconductive detectors each respond to electromagnetic energy of a different wavelength, wherein the two other resistors are configured for each exhibiting an electrical resistance essentially constant under illumination, wherein an external voltage is applicable to the sensor system, wherein the sensor system is configured for measuring a differential voltage, wherein the differential voltage is dependent on changes of the electrical resistances of the photoconductive detectors, wherein the differential voltage gives a mathematical ratio of the four respective resistances.

2. The sensor system according to claim 1, wherein the resistors exhibiting an electrical resistance essentially constant under illumination are photoconductive detectors, darkened by a cover.

3. The sensor system according to claim 1, wherein the resistors essentially constant under illumination are thermistors, wherein a change of their resistance as a function of temperature has the same characteristics as of the photoconductive detectors.

4. The sensor system according to claim 1, wherein the photoconductive detectors are covered by filter elements for preparation of light at different wavelengths.

5. The sensor system according to claim 1, wherein the resistors are interconnected by a bridge circuit arrangement.

6. The sensor system according to claim 1, wherein the sensor system is in direct line of sight of a measured object, wherein filters are arranged to be within the wavelength range of the electromagnetic radiation which is in the line of sight.

7. The sensor system according to claim 6, wherein the sensor system and the measured object are separated by a separating object, wherein the separating object is at least partially transparent at the at least two wavelengths, wherein the filters are arranged to be within the wavelength range of the electromagnetic radiation transmitted through the separating object.

8. The sensor system according to claim 1, wherein the four photoconductive resistors are arranged in an array next to another.

9. A method of using a sensor system according to claim 1 as a sensor for temperature measurement.

10. A method of using a sensor system according to claim 1 as a sensor for gas and/or liquid analysis.

11. A method of using a sensor system according to claim 1 as a sensor for concentration of gas and/or liquid or gases and/or liquids.

12. A method of using a sensor system according to claim 1 as a sensor for material classification between to pre-defined material classes.
